(19) United States
(12) Patent Application Publication
(10) Pub. No.: US 2012/0294883 A1
(43) Pub. Date: Nov. 22, 2012

(54) OLIGOMERIC RECEPTOR LIGAND PAIR MEMBER COMPLEXES

(76) Inventor: Nikolai Franz Gregor SCHWABE, Oxford (GB)

(21) Appl. No.: 13/420,573

(22) Filed: Mar. 14, 2012

Related U.S. Application Data
(63) Continuation of application No. 12/208,108, filed on Sep. 10, 2008, now abandoned.
Application No. 12/208,108 is a continuation of application No. 10/770,304, filed on Feb. 2, 2004, now abandoned.

(30) Foreign Application Priority Data
Oct. 30, 2003 (GB) 0325346.5

(51) Int. Cl. C07K 19/00; G01N 33/53; C12N 5/0783; C12P 21/00; A61K 39/00; A61P 37/04

(52) U.S. Cl. 424/192.1; 530/402; 435/375; 435/69.7; 435/7.21

(57) ABSTRACT

This invention concerns an oligomeric receptor-ligand pair member complex in general and an oligomeric MHC-peptide complex in particular and a method of labeling, detecting and separating mammalian T cells according to the specificity of their antigen receptor by use of the oligomer. The invention further concerns a method of targeting the oligomeric receptor-ligand pair member complexes to target molecules of the surface of a target cell in order to present antigens on the target cell. The invention further concerns related pharmaceutical and diagnostic compositions and processes.

REFERENCE TO RELATED APPLICATIONS

This application is a continuation of U.S. patent application Ser. No. 10/770,304, filed Feb. 2, 2004, which claims priority to GB 0325346.5, filed Oct. 30, 2003. This application may be considered related to co-pending, co-owned U.S. patent application Ser. No. 10/769,831, filed Feb. 2, 2004, which is a continuation-in-part of PCT Patent Application PCT/EP03/09056, filed on Aug. 14, 2003. This application also may be considered related to co-pending, co-owned U.S. patent application Ser. No. 10/770,140, filed Feb. 2, 2004. The contents of all these applications are incorporated by reference herein.

### FIELD OF THE INVENTION

The present invention relates to an oligomeric receptor-ligand pair member complex in general and an oligomeric MHC-peptide complex in particular and a method of labeling, detecting and separating mammalian T cells according to the specificity of their antigen receptor by use of the oligomer. The invention further relates to a method of targeting said oligomeric receptor-ligand pair member complexes to target molecules of the surface of a target cell in order to present antigens on the target cell, for example for stimulating mammalian T cells according to the specificity of their antigen receptor.

### BACKGROUND OF THE INVENTION

Major Histocompatibility Complex (MHC) molecules, which are found on the cell surface in tissues, play an important role in presenting cellular antigens in the form of short linear peptides to T cells by interacting with T cell receptors (TCRs) present on the surface of T cells.

It has been established that isolated or recombinant forms of MHC-peptide molecules are useful for detecting, separating and manipulating T cells according to the specific peptide antigens these T cells recognize. It has also been understood that the interaction between MHC molecules and TCRs across cell surfaces is multimeric in nature and that the affinity of a single MHC molecule for a given TCR is generally low in affinity.

As a consequence, there has been an effort to develop multimeric forms of isolated or recombinant MHC-peptide molecules to make such molecules more useful in the applications described above.

European Patent Application EP 812 331 discloses a multimeric binding complex for labeling, detecting and separating mammalian T cells according to their antigen receptor specificity, the complex having the formula (α-β-P)ₙ, wherein (α-β-P) is an MHC peptide molecule, n is ≧2, α comprises an α chain of a MHC class I or MHC class II class molecule, β comprises a β chain of an MHC protein (β₂ microglobulin for MHC class I) and P is a substantially homogeneous peptide antigen. The MHC peptide molecule is multimerised by biotinylating the C terminus of one of the α or β chain of the MHC molecule and coupling of MHC monomers to tetravalent streptavidin/avidin or by providing a chimeric protein of an MHC molecule which is modified at the C terminus of one of the α or β chain to comprise an epitope which is recognised by a corresponding antibody that serves as a multimerising entity. The document further teaches use of the MHC oligomers for detecting, labeling and separating specific T cells according to their TCR specificity.

European Patent Application EP 665 289 discloses specific peptides, MHC molecules binding these peptides, and oligomers obtained by crosslinking of the respective MHC molecules having the specific peptide bound to them. Oligomerisation is achieved by using chemical crosslinking agents or by providing MHC chimeric proteins comprising an epitope, which is recognised by an immunoglobulin such as IgG or IgM. The MHC molecules may comprise a label and may be used for labeling, detecting, and separating T cells according to their specific receptor binding, and may eventually be employed in therapy of humans.

WO 93/10220 discloses a chimeric MHC molecule, comprising the soluble part of an MHC molecule, which can be either class I or class II MHC fused to an immunoglobulin constant region. The MHC portion of the molecule comprises complementary α and/or β chains and a peptide is bound in the binding groove of the MHC molecules. Due to the presence of the dimeric immunoglobulin scaffold these chimeric MHC-Ig molecules undergo self-assembly into a dimeric structure.

In other research the oligomerisation domain of cartilage oligomeric matrix protein (COMP) has been used as a tool for multimerising several proteins in the past. COMP has been described and characterised by Efimov and colleagues (see e.g. Proteins: Structure, Function, and Genetics 24:259-262 (1996)). COMP is a pentameric glycoprotein of the thrombospondin family. Self-assembly of the protein to form pentamers is achieved through the formation of a five-stranded helical bundle that involves 64 N-terminal amino acid residues of the protein. The amino acid sequence of the oligomerisation domain has been disclosed by Efimov et al., FEBS Letters 341:54-58 (1994), which for rat COMP reads as follows: QGQIPLGGDLAPQMLRELQETNAALQDVRELLRQQVKEITFLKNTVMECDACGMQPA RTPGLSV [SEQ ID NO: 9], corresponding to amino acid residues 21-83 of rat COMP.

WO 00/44908 discloses chimeric proteins that contain anti-angiogenic portions of TSP-1, TSP-2, endostatin, angiostatin, platelet factor 4 or prolactin fused to a portion of the N-terminal region of human cartilage oligomeric matrix protein (COMP) thus allowing for the formation of pentamers. The document is predominantly concerned with exploiting the anti-angiogenic effect mediated by the resulting chimeric proteins. According to this disclosure the chimeric protein should promote correct folding of the TSP-domains contained therein, so that they better mimic the natural proteins than peptides that are based on the TSR sequence.

U.S. Pat. No. 6,218,513 discloses globins containing non-naturally occurring binding domains for creating oligomers of said globins. The COMP oligomerisation domain is one of the disclosed binding domains. The advantage seen from oligomerisation relates to increased half-life and hence better resistance against intravasal degradation as well as reduced extravasation of the oligomerised globin proteins, due to their increased size compared to monomeric globin proteins.

Holler et al., Journal of Immunological Methods 237:159-173 (2000), disclose the development of improved soluble inhibitors of FasL and CD40L based on oligomerised receptors comprising TNF-receptor family members fused to the constant region of IgG or the self-assembling domain of COMP. It is concluded there that increased affinity of oligomeric soluble chimeric receptors of the TNF-receptor family is not a general phenomenon. It is found that the affinity of such oligomeric chimeric receptors to their ligand depends on the specific receptor-ligand pair under consideration and this is shown to vary significantly even between closely related proteins.

WO99/64464 describes targeting of MHC molecules to a specific cell type by linking the molecules to a target-specific antibody molecule. Where the target cell is a diseased, foreign or malignant cell, this method may be used to promote the lysis of the target cell by T cells in the immune system. In this context it is possible to make use of pre-existing T cell responses by employing MHC-peptide complexes that are specific for endemic persistent infections found in many healthy individuals such as Epstein Barr Virus infections and Cytomegalovirus infections. Alternatively, the target cell may be used as an antigen-presenting cell, to promote the proliferation of specific T cell clones that may kill other diseased, foreign or malignant cells or afford protective immunity against such cells to the patient.

As an example MHC-anti-CD20 antibody complexes are disclosed as suitable for targeting MHC complexes to CD20-positive Daudi cells, which may be useful in killing CD20 positive Daudi Burkitt's lymphoma cells.

Following on from this work Savage et al. in British Journal of Cancer (2002) 86: 1336-1342 have shown that it is possible to elicit antigen-specific syngeneic T cell responses against viral and tumor derived antigens using an two-step targeting system wherein biotinylated recombinant HLA-class I peptide complexes loaded with relevant antigenic peptides were attached to the surface of B cells via an anti-CD20 single chain variable domain antibody-streptavidin fusion protein. Concerns remain, however, in this case regarding the potentially unacceptable toxicity and immunogenicity of using non-human content in the MHC-targeting complexes of the prior art, such as the streptavidin content in the assembly of the MHC-streptavidin-scFv complex, especially where therapeutic use in vivo is envisaged.

The non-prepublished international application PCT/EP03/09056 filed Aug. 14, 2003 and assigned to the present applicant discloses an oligomeric MHC complex comprising at least two chimeric proteins, said chimeric proteins comprising a first section derived from an MHC peptide chain or a functional part thereof and a second section comprising an oligomerising domain derived from an oligomer-forming coiled-coil protein, wherein formation of the oligomeric MHC complex occurs by oligomerisation at the oligomerisation at the oligomerising domain of the chimeric proteins, and wherein at least two of the first sections are derived from the same MHC peptide chain.

Further, the non-prepublished British national application GB 0323324.4 filed Oct. 6, 2003 and assigned to the present applicant discloses an oligomeric MHC complexes similar to those disclosed in PCT/EP03/09056, which are however additionally provided with an attachment means for targeting to target molecules on the surface of a target cell in order to stimulate mammalian T cells according to the specificity of their antigen receptor. The disclosures of both of these documents are incorporated herein by way of reference in their entirety. In each of these documents, however, the MHC molecules are fused through at least one of their polypeptide chains to the oligomerising domain.

The attempts for multimerisation of MHC proteins described hereinbefore pose several disadvantages. Chemical crosslinking for example typically results in a non-predictable structure of the final MHC oligomer, which may vary considerably for each complex. Hence, binding to the target may vary likewise, depending on the final oligomer structure. This in turn can impede accuracy and reliability of any assay system the oligomers are used in. In the worst case chemical crosslinking may even prevent formation of a functional MHC oligomer altogether.

Fusing either one or two of the MHC polypeptide chains with the constant region of an immunoglobulin molecule such as described in WO 93/10220 results in a dimeric MHC molecular complex. Although the dimeric interaction can contribute to increasing the affinity of the complex, further multimerisation through anti-idiotypic anti-bodies or protein A or G may be required to reach the affinity required for various applications, such as detecting antigen specific T cells or activating such cells successfully.

Multimerisation of the MHC monomers by use of non-human binding partners such as the biotin/streptavidin binding pair or non-human antibodies on the other hand introduces non-human protein components into the oligomer. This raises concerns with regard to potential toxicity of the complex and/or immune responses against this non-human part of the complex in applications where in vivo use is envisaged, e.g. in human therapy. Accordingly it would be desirable to avoid non-human portions in the oligomeric complex.

In addition producing MHC multimers that rely on the biotin-streptavidin interaction involves a biotinylation reaction that can lead to significant loss of active material. Further, controlling the biotinylation efficiency of monomeric MHC subunits and quality of the final multimeric product is difficult. Finally, the tetrahedral arrangement of the biotin/streptavidin-complex puts certain sterical constraints and limitations on the obtained MHC multimer.

The MHC oligomers available from the art provide for a certain enhancement of affinity of the complex when compared to the MHC monomer itself. A further increase in affinity would, however, be very desirable without increasing the complexity of the synthesis of the complexes while assuring that such synthesis will yield molecules with high uniformity.

The prior art approaches of multimerising other receptor-ligand pair members by fusing one of their polypeptide chains to the oligomerising domain of COMP creates the difficulty that a relatively complicated fusion construct has to be created for each receptor-ligand pair member. In addition the full oligomeric receptor-ligand pair member complex has to assemble in one step, which may cause difficulties with the manufacturing process for certain receptor-ligand pair members. In such cases it would be desirable to have a means of separating the step of manufacturing the receptor-ligand pair member from that of oligomerising the same.

It is an object of the present invention to provide an oligomeric receptor-ligand pair member complex that is easy to manufacture in a generic manner. Specifically, it is an object to provide such a complex which provides improved flexibility with respect to the choice of the specific receptor-ligand binding pair member to be oligomerised over the prior art, that has a uniformly high valency of the receptor-ligand pair members which are available simultaneously for binding to their complementary receptor-ligand binding pair member. It is another object of the invention to produce such oligomeric receptor-ligand binding pair member complexes with a protein sequence that minimizes non-human content, as appropriate. Yet another object of the present invention is to provide such oligomeric receptor-ligand binding pair member complexes that can be targeted to the surface of target cells with high affinity and specificity.

### SUMMARY OF THE INVENTION

In its first aspect the present invention comprises an oligomeric receptor-ligand pair member complex comprising
(i) an oligomeric core, said core comprising at least two chimeric proteins, said chimeric proteins comprising a first section including at least one domain forming part of a first member of a complementary binding pair
   and a second section comprising an oligomerising domain derived from an oligomer-forming coiled-coil protein, wherein formation of the oligomeric core occurs by oligomerisation at the oligomerising domain of the chimeric proteins; and
(ii) at least two receptor-ligand pair member peptides derived from a receptor-ligand pair member peptide chain or a functional part thereof, wherein each receptor-ligand pair member peptide further comprises attached thereto a second member of said complementary binding pair capable of binding to the first complementary binding pair member as defined in (i);
   and wherein each receptor-ligand pair member peptide is bound to the core via binding of the first and second members of the complementary binding pair;
   and in which complex at least two of the receptor-ligand pair member peptides are derived from the same receptor-ligand pair member peptide chain.

In a preferred embodiment of the invention the oligomerising domain comprised in the second section in at least one of the chimeric proteins is derived from the pentamerisation domain of the human cartilage oligomeric matrix protein (COMP). Preferably the oligomerising domain comprises and preferably consists of the amino acids 1 to 128, preferably 20 to 83, most preferably 20 to 72 of COMP.

Preferably the first section in at least one of the chimeric proteins comprises one or more immunoglobulin-derived domains, which are selected from the group consisting of a Fab fragment, a V_{L} domain, a V_{H} domain.

More preferably, the oligomeric receptor-ligand pair member complex may additionally comprise the complementary variable (and constant) domains of respective immunoglobulin domains.

Yet more preferably, the first section comprises a single chain variable fragment.

The oligomeric receptor-ligand pair member complex may additionally comprise a linker between the first section and the second section in at least one of the chimeric proteins.

In the oligomeric receptor-ligand pair member complex of the invention the complementary binding pair member as recognized by the binding pair member, of which the domain comprised in the first section in the chimeric proteins forms part of as defined in (i) above forms part of, may further be covalently attached to a receptor-ligand pair member peptide chain.

Additionally, the second member of the complementary binding pair may be a peptide fused to the receptor-ligand pair member peptide, preferably at its C terminal end.

In a second aspect of the invention the oligomeric receptor-ligand pair member complex of the invention further comprises attaching means for selectively attaching said receptor-ligand pair member complex to a target cell.

Preferably the said attaching means is comprised in a third section of at least one of the chimeric proteins of the complex.

In a preferred embodiment said attaching means comprises a linking polypeptide with high specific affinity for a target cell specific molecule on the surface of the target cell.

More preferably said linking polypeptide is also a single chain variable fragment derived from a monoclonal antibody.

Optionally, at least one of the chimeric proteins further comprises one or more domains selected from the group consisting of a further linker, a tagging domain and a purification domain.

Preferably the first section of the chimeric protein is located N-terminal of the second section and the third section, if present, is located C-terminal of said second section.

In another preferred embodiment of the invention the chimeric protein has the structure
scFv-linker-COMP-linker-AA
wherein
scFv is the single chain variable fragment, preferably V_{H}-linker-V_{L};
COMP is the oligomerisation domain of COMP;
linker means a peptide linker; and
AA is a peptide selected from one or more domains selected from the group consisting of a further linker, a tagging domain, a purification domain and a linking polypeptide with high specific affinity for a target cell specific molecule on the surface of the target cell.

In a further preferred embodiment at least one of the peptides derived from the receptor-ligand pair member or functional part thereof is an MHC peptide or functional part thereof.

Preferably peptides derived from the receptor-ligand pair member peptides or functional part thereof in the oligomeric receptor-ligand pair member complex are derived from the extra-cellular part of the MHC class I or II α chain or the extra-cellular part of the MHC class I or II β chain. More preferably the oligomeric receptor-ligand pair member complex further comprises complementary MHC peptide chains to at form least two functional MHC binding complexes.

More preferably the oligomeric receptor-ligand pair member complex further comprises a peptide bound to the MHC portions of the complex in the groove formed by the MHC α1and α2 domains for class I complexes or the MHC α1 and α1 domains for class II complexes. Most preferably the peptide is substantially homogeneous.

In a preferred embodiment of this MHC complex the peptide is selected from the group consisting of (a) a peptide against which there is a pre-existing T cell immune response in a patient, (b) a viral peptide e.g. occurring in the group of Epstein Barr Virus, Cytomegalovirus, Influenza Virus, (c) an immunogenic peptide of immunogens used in common vaccination procedures, and (d) a tumour specific peptide, a bacterial peptide, a parasitic peptide or any peptide which is exclusively or characteristically presented on the surface of diseased, infected or foreign cell.

Optionally the oligomeric receptor-ligand pair member complex according to one of the preceding claims comprising a label.

In a third aspect of the invention the oligomeric receptor-ligand pair member complex is used to amplify antigen-specific or allospecific T cells in vivo or in vitro.

A fourth aspect of the invention concerns a pharmaceutical or diagnostic composition, comprising an oligomeric receptor-ligand pair member complex according to the invention, optionally in combination with a pharmaceutically acceptable carrier.

Here, an oligomeric MHC complex of the invention can be used for preparing a pharmaceutical composition for immunizing a patient against a disease or condition which is characterized by the presence in the patient's body of cells displaying disease associated MHC binding peptides on the surface thereof that are associated with the said disease or condition and wherein the oligomeric MHC complex comprises binding peptides which are similar to or substantially the same as the said disease associated MHC binding peptides.

As a consequence such oligomeric receptor-ligand pair member complexes of the invention may be used for the preparation of a pharmaceutical composition for causing an immune response in a patient to destroy unwanted target cells by directing an immune response against said target cells.

In a fifth aspect the present invention concerns a method of preparing an oligomeric receptor-ligand pair member complex as described herein, said method comprising the steps of:
a) constructing a vector comprising the recombinant expression cassette comprising a promoter sequence operably linked to a nucleotide sequence coding for a chimeric protein which chimeric protein comprises a first section comprising at least one domain forming part of a first member of a complementary binding pair and a second section comprising an oligomerising domain derived from an oligomer-forming coiled-coil protein, wherein oligomerisation occurs by alignment of at least two substantially identical versions of the polypeptide chain from which the oligomerising domain is derived; expressing said vector in an appropriate host, and recovering the expressed chimeric protein;
b) oligomerising said chimeric protein by alignment of the oligomerisation domains to form a complex core;
c) providing a receptor-ligand pair member peptide derived from an receptor-ligand pair member peptide chain or a functional part thereof, which receptor-ligand pair member peptide further comprises attached thereto a second member of the said complementary binding pair and capable of binding to the first complementary binding pair in the first section of the chimeric protein; and
d) attaching the receptor-ligand pair member peptide by binding of the first and second members of the complementary binding pair to the core.

In a sixth aspect the invention concerns an oligomeric core for forming an oligomeric receptor-ligand pair member complex of the invention wherein said core comprises at least two chimeric proteins, said chimeric proteins comprising a first section including at least one domain forming part of a first member of a complementary binding pair and a second section comprises an oligomerising domain derived from an oligomer-forming coiled-coil protein, wherein formation of the oligomeric core occurs by oligomerisation at the oligomerising domain of the chimeric proteins, wherein the second member of the complementary binding pair is selected from the group of a polypeptide epitope tag of less than 50 amino acids in length, a polypeptide incorporating a post-translational modification.

A seventh aspect of the invention concerns a method of labeling and or detecting a cell population in a sample according to the specificity of a complementary receptor-ligand pair member present on the surface of cells in the cell population, the method comprising

combining an oligomeric receptor-ligand pair member complex according to the invention and a suspension or biological sample comprising the cell population, and detecting the presence of specific binding of said complex and the cells in said population.

An eighth aspect of the invention concerns a method of separating a cell population in a sample according to the specificity of a complementary receptor-ligand pair member present on the surface of cells in the cell population, the method comprising combining an oligomeric receptor-ligand pair member complex according to the invention and a suspension or biological sample comprising the cell population, and separating cells in the cell population bound to said complex from unbound cells.

Preferably in the seventh and eighth aspect said cell population is a T cell population or a NK cell population.

In a further preferred embodiment according to the seventh and eighth aspect of the invention at least one of the peptides derived from the receptor-ligand pair member or functional part thereof is an MHC peptide or functional part thereof. More preferably the peptides derived from the receptor-ligand pair member peptides or functional part thereof in the oligomeric receptor-ligand pair member complex are derived from the extra-cellular part of the MHC class I or II α chain or the extra-cellular part of the MHC class I or II β chain. Yet more preferably the oligomeric receptor-ligand pair member complex further comprises complementary MHC peptide chains to at form least two functional MHC binding complexes. Yet more preferably the oligomeric receptor-ligand pair member complex further comprises a peptide bound to the MHC portions of the complex in the groove formed by the MHC α1 and α2 domains for class I complexes or the MHC α1 and β1 domains for class II complexes. Most preferably the peptide is substantially homogeneous.

### DESCRIPTION OF THE DRAWINGS

FIG. 1 is a schematic drawing of an oligomeric receptor-ligand pair member complex of the invention, wherein the receptor-ligand pair member selected is a MHC class I complex of the invention. The figure shows two out of several subunits of the fully assembled oligomeric class I chimeric complex. As shown β2-microgobulin (β2m) is fused to an epitope tag (E) at its C-terminus. The epitope tag may be separated from the C-terminus of β2m by an appropriate linker (not shown) such as a serine glycine linker. A single chain variable fragment scFv of a monoclonal antibody specific to the epitope tag (E) is fused to the coiled oligomerisation domain of COMP as an oligomer-forming coiled-coil protein and spaced from this domain by a second linker (L2). The scFv contains N-terminal to C-terminal the variable heavy chain V_{H} fused to the variable light chain of the antibody V_{L}. V_{H} and V_{L} are separated by a first linker (L1) A further linker (L3) is provided at the C terminal end of the oligomerising domain, followed by a recognition sequence (BP) for biotinylation by biotin-protein ligase BirA and finally a tagging/purification domain (TD) for purification and/or detection. A biotin molecule (bt), attached to the Lysine residue of the recognition sequence for biotinylation (BP) is also shown. The complementary MHC class I α chain having the domains α1, α2, and α3, is assembled with β2m and antigenic peptide (P). Disulphide bonds (S—S), stabilizing the MHC portion of the complex are indicated. Further disulphide bonds are also shown, illustrating the specific case where the oligomerising domain is derived from COMP, which will assemble into a stable pentamer, wherein these disulphide bonds link each pair of the COMP domains in the pentamer. The amino and carboxyl termini of the respective protein chains in the complex are indicated by N and C, respectively. In an alternative embodiment, however the fusion between the scFv and the oligomerisation domain may be C-terminal to N-terminal.

FIG. 2 is a schematic drawing of an oligomeric MHC class II complex of the invention. The figure generally follows the nomenclature of FIG. 1 with the difference that β2m is replaced by the MHC class II α chain, having the domains α1, α2, fused to the epitope tag E and the MHC class I α chain is replaced with the MHC class II β chain. Here the α chain is assembled with its complementary MHC class II β chain, having the domains α1, α2, and the peptide P.

FIG. 3 shows a schematic drawing of a portion of the oligomeric receptor-ligand pair member complex of the invention, illustrating optional alternatives the first section comprising the domain forming part of the first member of the complementary binding pair.

In FIG. 3a the V_{H} and V_{L} domains of the scFv are interchanged. Optionally also portions of the heavy and light chain constant domains of the F(ab) fragment of the monoclonal antibody may be included in the construct (not shown).

In FIG. 3b only the variable domain of the light chain V_{L} is fused to the oligomerisation domain and V_{H} is associated non-covalently with V_{L}, which may either occur during protein expression or through a subsequent refolding step. Again V_{H} and V_{L} may be interchanged as well.

FIG. 3c shows the Epitope tag E fused directly to the oligomerising domain in the complex core via the linker L2. In this scenario the complementary scFv or immunoglobulin portion from the monoclonal antibody will be attached or fused to the receptor-ligand pair members of the complex. In the case where the complementary scFv or immunoglobulin portion is fused to the receptor-ligand pair members of the complex, such fusion may occur between any of the termini of the protein chains of the receptor-ligand pair members of the complex and any of the termini of the protein chains of the complementary scFv or immunoglobulin portion from the monoclonal antibody.

FIG. 4a shows schematic drawing of a portion of the oligomeric receptor-ligand pair member complex of the invention enabling use of the complex to be targeted with high specific affinity to a target cell specific molecule on the surface of the target cell. As shown a further linker (L3) is provided at the C terminal end of the oligomerising domain, followed by a scFv comprising the variable region of the heavy chain V_{H} of an immunoglobulin molecule which is specific for a desired cell-surface molecule. V_{H} is linked at its C-terminal end to the N-terminus of the variable region of the immunoglobulin light chain V_{L} via a fourth linker (L4).

In FIG. 4b the heavy and light chains of the immunoglobulin molecule are inter-changed.

In FIG. 4c, both the variable and constant domains of the binding portion of the immunoglobulin molecule are included in the construct and the linking can occur between the constant region of the first chain and the variable region of the second chain.

In FIG. 4d the linker between the immunoglobulin heavy and light chains is omitted and the final complex is formed by non-covalent assembly of the immunoglobulin chains, either during protein expression or by means of a separate refolding reaction.

FIG. 5 shows simultaneous multivalent binding of the oligomeric MHC-complex of the invention to a target cell via a target molecule present on the surface of the target cell, for which the attachment portion of the oligomeric-MHC complex is specific, and to the T cell receptors on the surface of an antigen specific T cell which engage the binding portion of the MHC-peptide complex. The domain structure of the T cell receptors is also indicated, including disulphide bonds stabilizing these molecules.

### DETAILED DESCRIPTION OF THE INVENTION

The oligomeric receptor-ligand pair member complex of the invention allows for overcoming the disadvantages and drawbacks of the prior art. More specifically, the inventors have found that the above oligomeric receptor-ligand binding pair member complex can have a very high affinity to its complementary receptor-ligand pair member, while offering the convenience of a generic two step assembly process of binding the receptor-ligand binding pair member to the oligomeric core.

For example oligomeric MHC-peptide complexes can be generated that have a higher affinity than an oligomer obtained by e.g. tetramerising the MHC complexes by coupling through biotin and streptavidin described in EP 812 331 or scFv-streptavidin as in Savage et al. in British Journal of Cancer (2002) 86, 1336-1342.

Without wishing to be bound by theory it is believed that this increase in affinity is achieved when three or more receptor-ligand pair member molecules are arranged substantially in the same plane with all binding faces oriented in the same direction. The complex according to the invention further allows for effective labeling while minimizing the interference of the chosen label with the active receptor-ligand pair member domains, as the label(s) are located on the opposite end of the oligomer-forming coiled-coil domain.

In the same manner the cell surface attachment portions of the oligomeric complex where applicable can all disposed at the same end of the complex allowing for highly multivalent binding to target molecules that are present on the surface of target cells.

The meaning of a “receptor-ligand pair” within the scope of the present invention encompasses any two ligands with a specific binding affinity for one another of which at least the member to be oligomerised in the oligomeric core of the complex of the invention comprises protein content. As a consequence it includes any two binding proteins with a specific affinity for one another. Each individual partner of the pair is referred to as a “receptor ligand pair member”.

Possible receptor-ligand binding pair members that could be oligomerised to form the complexes of the invention could include, without limitation, for example other molecules of the immune system, such as T cell receptors, TNF family ligands, TNF family receptors, Fc receptors, interleukins and their receptors, and in general CD markers and their ligands, for example CD4, CD8, B7.1 and its ligands, B7.2 and its ligands CD40, C40L, apoptosis inducing receptors or ligands, such as FAS and FASL, molecules of the Major Histocompatibility Complex (MHC), such as Class I and Class II MHC-peptide complexes, Non-classical MHC molecules, Minor Histocompatibility Antigens, lectins, protein NG, immunoglobulin molecules, B cell receptors and ligands, NK cell receptors and ligands, and cell surface signaling receptors and ligands.

The meaning of an “oligomer-forming coiled-coil protein” within the scope of the present invention is a protein comprising an oligomerising domain. Said domains comprise two or more polypeptide subunits that may or may not be identical. The subunit of two or more of such oligomerisation domains assemble with one another such that each subunit in the oligomerising domain assumes a substantially helical conformation in the assembled state, wherein the subunits in the oligomerisation domain are arranged along an identifiable axis, wherein the oligomerising domain has two identifiable opposite ends along said axis, and wherein at least two of the polypeptide subunits have the same amino-to-carboxyl orientation along said axis. Corresponding oligomerisation domains and oligomer-forming coiled-coil proteins are known in the art as e.g. cited in the introductory portion hereof.

The complementary binding pair described above, which is used for binding the receptor-binding pair members to the oligomeric core may be an epitope-receptor pair or hapten-receptor pair such as an epitope- or hapten-antibody pair. The epitope may be a short amino acid sequence, as recognized by a suitable monoclonal antibody with high affinity. Examples for suitable epitope receptor pairs include PK tag (GKPIPNPLLGLDST) [SEQ ID NO: 1], c-Myc tag (EQKLISEEDL) [SEQ ID NO: 2], HA tag (YPYDVPDYA) [SEQ ID NO: 3] (amino acid sequences indicated in single letter code) and monoclonal antibodies raised against these tags, and calmodulin and calmodulin binding peptide. Further complementary protein sequences could be used that are derived from heterodimeric leucine zipper proteins such as the Fos and Jun DNA binding proteins. Here sections of these proteins would be chosen that form stable heterodimers according to methods well known in the art. Alternatively one or both members of the binding pairs may be introduced to a protein backbone through post-translational modifications of the backbone.

Preferably the complementary binding pair will have a short epitope tag, such as the epitope tags described above as a first member and F(ab) fragment, or a single chain variable fragment (scFv) derived from a monoclonal antibody specific which is specific to the epitope tag as a first member.

For example, enzymatic modifications may be carried out by modifying an enzyme recognition sequence fused to one of the termini of the chimeric protein or a receptor-ligand pair member peptide chain. Modifying enzymes of interest may include BirA, glycosylases, protein transferases, protein kinases, carboxy peptidases and the like. The subunit may be reacted with the modifying enzyme to introduce e.g. biotin, sugar, phosphate, farnesyl, or other modifications, which provide a complementary binding pair member or a unique site for further modification, such as chemical cross linking that will provide a complementary binding pair member. An alternative strategy is to introduce an unpaired cysteine to the subunit thereby introducing a unique and chemically reactive site for binding. Any such modification will be at a site in the receptor-ligand pair member or the chimeric protein that will not impair its other functional characteristics. Suitable enzyme recognition sequences for biotinylating peptides with BirA are referenced in EP 812 331.

The term “domain forming part of a first complementary binding pair member” as used herein has the meaning of a polypeptide sequence that forms at least a portion of the first complementary binding pair member. For example where the first complementary binding pair member is an enzymatically biotinylated peptide the domain would be the enzyme recognition sequence. In another example where the first complementary binding pair member is an antibody fragment, such as a F(ab) fragment, the domain would include at least one of the immunoglobulin domains of the F(ab) fragment, whereas the remaining immunoglobulin domains may be associated non-covalently with this domain. In another specific case the said domain may be an unpaired cysteine residue, which can bind to a sulfuhydryl reactive group forming part of the second complementary binding pair member.

The oligomerising domain may be derived from a suitable oligomer-forming coiled-coil protein of any species. Preferably, oligomerising domain is derived from a human version oligomer-forming coiled-coil protein, in which case unwanted immune responses and/or rejection reactions are minimised in situations where the complex is to be administered to humans.

Examples for oligomer-forming coiled-coil proteins include various types of collagen, triple coiled-coil domains of C-type lectins, such as mannose binding protein (MBP); C1q, myosin, leucine zippers such those occurring in p53, GCN4, bacteriophage P22 Mnt repressor; and the trombospondin family proteins such as COMP. Preferably the oligomerisation domain is derived from the cartilage oligomeric matrix protein COMP. More preferably, the oligomerising domain is derived from the human version of COMP for the reasons described above.

The number of chimeric proteins (n) comprised in the oligomeric core of the oligomeric receptor-ligand pair member complex of the invention will typically depend on the type of oligomerisation domain the second section of the chimeric proteins is derived from and can in general be 2 or more, preferably n=2 to 10, most preferably n=3 or 5. If the second section of the chimeric proteins to be oligomerised is e.g. derived from the pentamerisation domain of the COMP this number will typically be five such that the oligomer will be a pentamer (n=5), whereas in case these oligomerisation domains are derived from collagen this number will be three (n=3).

The term “chimeric protein” as used herein means a single peptide protein, the amino acid sequence of which is derived at least in part from two different naturally occurring proteins or protein chain sections, in this case the first section including at least one domain forming part of a first member of a complementary binding pair the second section comprising a peptide substantially comprising at least a significant proportion of an oligomerising domain. With the term “a functional part thereof” as used herein, a part of a peptide chain is meant, which still exhibits the desired functional characteristics of the full-length peptide it is derived from.

According to a first embodiment a MHC peptide chain in the complex is the extra-cellular part of the MHC class I or II α chain. According to another embodiment the MHC peptide chain is the extra-cellular part of an MHC class I or II β chain. These may be assembled with their complementary MHC peptide chains, respectively. With the term “complementary” MHC peptide chain the respective other peptide chain of a naturally occurring MHC complex is meant. The complementary chain to an α chain of the MHC complex is the β chain and vice versa. The MHC proteins may be from any vertebrate species, e.g. primate species, particularly humans; rodents, including mice, rats, hamsters, and rabbits; equines, bovines, canines, felines; etc. Of particular interest are the human HLA proteins, and the murine H-2 proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin. Included in the murine H-2 subunits are the class I H-2K, H-2B, H-2Q, H-2D, and the class II I-Aα, I-Aβ, I-Eα and I-Eβ. Amino acid sequences of some representative MHC proteins are referenced in EP 812 331.

In addition the MHC peptide chains in the complex may also be derived from the extracellular parts of non-classical MHC molecules, such as HLA-E, HLA-F, HLA-G, Q1-A and CD1.

In a preferred embodiment, the MHC peptide chains correspond to the soluble form of the normally membrane-bound protein. The soluble form is derived from the native form by deletion of the transmembrane and cytoplasmic domains. For class I proteins, the soluble form will include the α1 , α2 and α3 domains of the αchain and β2-microglobulin. For class II proteins the soluble form will include the α1 and α2 or β1 and β2 domains of the α chain or β chain, respectively.

For class I subunits, not more than about 10, usually not more than about 5, preferably none of the amino acids of the transmembrane domain will be included. The deletion may extend as much as about 10 amino acids into the α3 domain. Preferably none of the amino acids of the α3 domain will be deleted. The deletion will be such that it does not interfere with the ability of the α3 domain to fold into a functional disulfide bonded structure. The class I β chain, β2m, lacks a transmembrane domain in its native form, and does not have to be truncated. Generally, no class II subunits will be used in conjunction with class I subunits.

The above deletion is likewise applicable to class II subunits or the domain forming part of the first member of the complementary binding pair. It may extend as much as about 10 amino acids into the α2 or β2 domain, preferably none of the amino acids of the α2 or β2 domain will be deleted. The deletion will be such that it does not interfere with the ability of the a2 or β2 domain to fold into a functional disulfide bonded structure.

One may wish to introduce a small number of amino acids at the polypeptide termini, usually not more than 25, more usually not more than 20. The deletion or insertion of amino acids will usually be as a result of the requirements in cloning, e.g. as a consequence of providing for convenient restriction sites or the like, and to manage potential steric problems in the assembly of the molecules. In addition, one may wish to substitute one or more amino acids with a different amino acid for similar reasons, usually not substituting more than about five amino acids in any one domain.

In an alternative embodiment the MHC class I or class II α and β chains may be joined as a single chain construct via an appropriate linker, such as a glycine-serine linker. The construct may be α-linker-β in the direction of N-terminus to C-terminus or β-linker-α in the direction of N-terminus to C-terminus, whichever is more convenient for the application in question.

According to the present invention the first section including at least one domain forming a first member of a complementary binding pair is fused to the oligomerisation domain of an oligomer-forming coiled-coil protein. Where the pentamerising domain of COMP is used as the oligomerising domain, this domain comprises and more preferably consists of the N terminal amino acids 1 to 128, preferably 20 to 83, most preferably 20 to 72 of said protein as discussed in the prior art section of this invention disclosure. With regard to numbering of amino acids, reference is made to Efimov et al., FEBS Letters 341:54-58 (1994). Further, similar to the MHC part of the complex and the domain forming part of the first member of the complementary binding pair comprised in the first section of the chimeric protein of the invention, this domain can be altered by amino acid substitution, deletion or insertion, as long as the self-assembly of the oligomerising domain is not impaired.

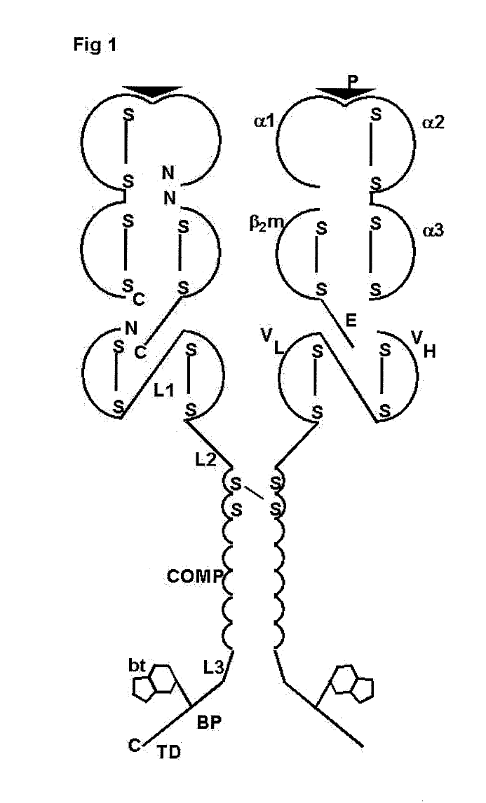

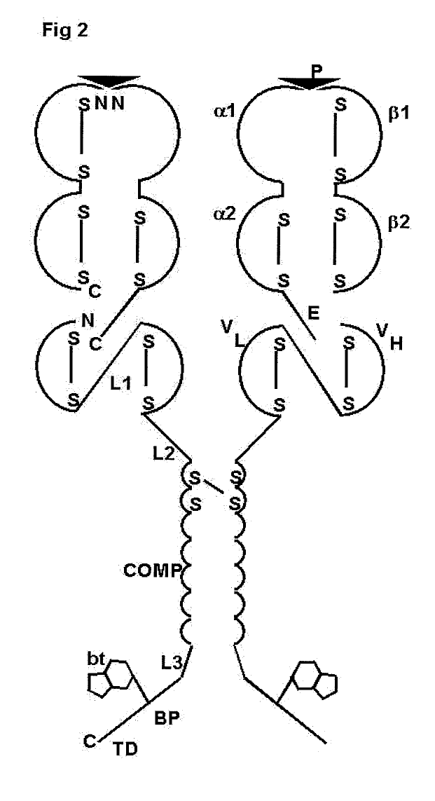

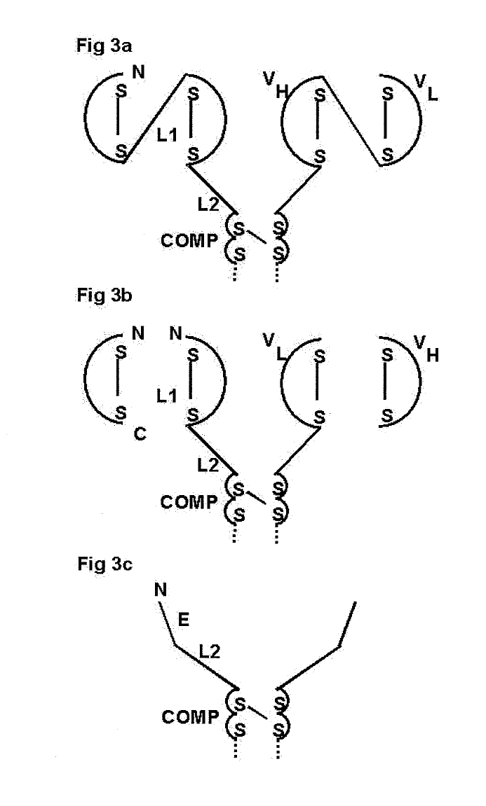

Fusion of both peptide chains, the first section and the second section including the oligomerising domain, respectively, may be direct or, as is shown in FIGS. 1 and 2, may include a linker (L2) connecting and spacing apart the MHC peptide and the oligomerising domain (COMP) in the chimeric protein. Joining by use of a linker is preferred. In general such linker will comprise not more than 30, preferably not more than 26 amino acids. Suitable linkers are known in the art and include e.g. immunoglobulin hinge regions, or serine glycine repeat sequences.

As is further shown in FIGS. 1 and 2, the chimeric protein of the invention may further include, preferably at its C terminal end, one or more of a second linker (L2), a biotinylation peptide (BP) and a tagging domain and/or a purification domain (TD) in either order. In a preferred embodiment the chimeric protein of the invention includes all three of the above domains in this order. In general the second linker will comprise not more than 25, preferably not more than 20 amino acids and can be the same as detailed for the first linker above.

The purification domain optionally to be included in the chimeric protein of the invention can be any domain assisting in purification of the complexes of the invention and their subunits, e.g. by providing specific binding characteristics. Appropriate sequences are known to the skilled worker and can be applied as long as they do not interfere with the functional domain forming part of the first complementary binding pair member and/or the receptor ligand pair members and oligomerising domains of the chimeric protein. Preferably the purification domain is a hexahistidine sequence.

According to the second aspect of the invention, the oligomeric receptor-ligand pair member complex of the invention formed by oligomerising the appropriate number of chimeric proteins as defined above may further comprise an attaching means for selectively attaching (and targeting) said complex (which preferably is an oligomeric MHC complex) to a target cell. The attaching means can in general be affixed to the complex at any suitable site and by any suitable means, provided it does not interfere with the oligomerisation, and the receptor-ligand binding, and does not separate under conditions of its use. Typically the attaching means will thus be affixed to the complex by covalent bonding either directly or through a (polypeptide or other) linker, e.g. conventional hydrocarbon spacers, or through specific binding pairs, such as antibody-epitope reactions or antibody-hapten reactions.

Preferably the attaching means is affixed to an oligomerisation domain of one or more, and preferably to each one of the chimeric proteins. Again this affixation may be by any appropriate means but preferably occurs through a peptide bond or a polypeptide linker. Most preferably in such an embodiment the attaching means will thus be itself a linking peptide. It may in this case be comprised in and preferably form a third section of the chimeric protein of the invention.

This third section may be attached to either the N- or C-terminal end of the oligomerisation domain, and is preferably attached to the opposite end with regard to attachment of the first section.

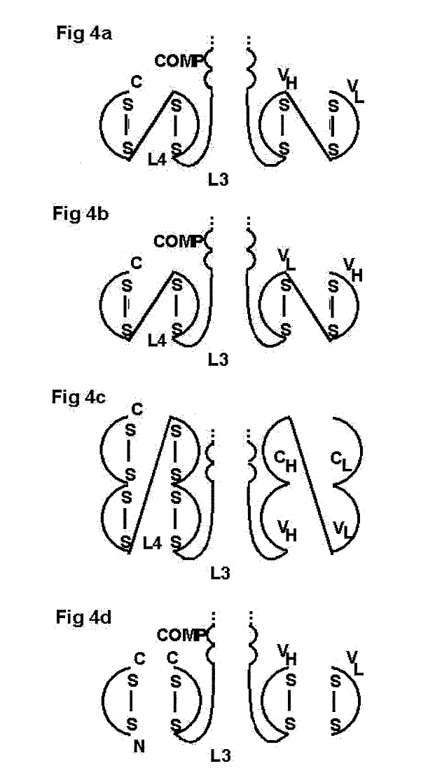

In a preferred embodiment, as is shown in FIG. 4, the chimeric protein of the invention is further fused, preferably at the C terminal end and optionally via a third polypeptide linker (L3) such as a Glycine-Serine linker, e.g. (GGGGS)₅ [SEQ ID NO: 4], to a linking polypeptide with a high specific affinity for a cell surface molecule on a target cell. Examples for suitable linking peptides are portions of the polypeptide chain of an antibody binding domain such as the immunoglobulin variable region light chain V_{L} or variable heavy chain V_{H}, which may be associated with their natural immunoglobulin domain counterparts, or an antibody fragment such as a single chain variable fragment (scFv) which is capable of binding specifically to target molecules on the surface of target cells, such as cell surface receptors. Presence of the linking polypeptide allows the chimeric proteins and complexes of the invention to be targeted to cell surfaces in vivo and in vitro for research, diagnostic and therapeutic purposes.

Again, one or more of the chimeric proteins of the complex that include the attachment means as described above may optionally further include, preferably at its C terminal end, one or more of a fourth linker (L4), a biotinylation peptide (BP) and a tagging domain and/or a purification domain (TD) in either order. In a preferred embodiment the chimeric protein of the invention includes all three of the above domains in this order. In another embodiment these domains may be present on differing chimeric proteins in the oligomeric receptor-ligand pair member complex. In general the fourth linker will comprise not more than 25, preferably not more than 20 amino acids and can be the same as detailed for the first linker above.

Where a purification domain is to be optionally included in one or more of the chimeric proteins of the complex the skilled worker will know of several possible sequences, including, e.g. a hexahistidine sequence.

The tagging domain optionally included in one or more of the chimeric proteins of the complex can be any domain which allows for labeling of the protein. Preferably the tagging domain or the chimeric protein includes a label. This label can be included in the domain itself such as an epitope recognised by an antibody or a light detectable or radioactive label. Preferably, the label is selected from the group consisting of fluorescent markers, such as such as FITC, phycobiliproteins, such as R- or B-phycoerythrin, allophycocyanin, Cy3, Cy5, Cy7, a luminescent marker, a radioactive label such as ¹²⁵1 or ³²P, an enzyme such as horseradish peroxidase, or alkaline phosphatase e.g. alkaline shrimp phosphatase, an epitope, a lectin or biotin/streptavidin.

Where the label is itself a protein, the polypeptide chain of the protein used for labeling can be fused to the chimeric protein in question, preferably at its C terminus, to the label protein. For example a fluorescent protein such as a green fluorescent protein (GFP), or a subunit of a phycobiliprotein could be used. GFP chimeric protein technology is well known in the art. Chimeric proteins comprising a suitable domain from a phycobiliprotein is described, for example in WO 01/46395.

Alternatively the label may be attached at a specific attachment site provided in the tagging domain. For example, the tagging domain may include a recognition site for the biotin protein ligase BirA to allow for site-specific biotinylation of the tagging domain and hence recognition by streptavidin/avidin. Suitable recognition sequences for BirA are well known in the art. Similarly a lectin may be attached, or any other site-specific enzymatic modification may be made by means of incorporating an amino acid recognition sequence for a modifying enzyme into the amino acid sequence of the chimeric proteins of the invention. Other possible types of enzymatic modification are described in EP 812 331. Alternatively labeling can be achieved by binding of a suitably labeled anti-body, or antibody fragment, such as a labeled F(ab) fragment to an epitope on the tagging domain or elsewhere on the molecule.

According to the first aspect thereof the present invention relates to an oligomeric receptor-ligand pair member complex formed by binding receptor-ligand pair members to an oligomeric core. Oligomerisation of the oligomerising domain in the chimeric protein typically occurs spontaneously and results in stable oligomers of the chimeric proteins, yielding the oligomeric core. Typically, the oligomeric complex will consist of several identical monomeric subunits, which are formed by binding identical receptor-ligand-pair members to the oligomeric core comprising identical chimeric proteins. If desired, however, two or more different receptor-ligand pair members which are all linked to the same second complementary binding pair member may be admixed in the oligomerisation step. Thereby heterogeneous oligomers may be obtained. Similarly heterogeneous oligomers can be formed by forming an oligomeric core from chimeric proteins whose first sections comprise domains forming part of different first members of differing complementary binding pairs. Generally, however, at least two of the chimeric proteins in each complex will comprise a first section derived comprising a domain forming part of the same complementary binding pair member. For n>2 the oligomer may contain at least two of the chimeric proteins in each complex which comprise a first section derived from the same complementary binding pair member. Preferably all chimeric proteins of the complex comprise a first section with a domain forming part of the same complementary binding pair member.

If the receptor-ligand pair member peptide is an MHC peptide, the oligomeric MHC complex may further comprise the complementary MHC peptide chain(s) to form functional MHC binding complexes as discussed above and may also comprise a peptide bound in the groove formed by the MHC α1 and α2 domains for class I MHC complexes or the MHC α1 and β1 domains for class II MHC complexes. Preferably the peptide is substantially homogeneous.

The antigenic peptides will be from about 6 to 14 amino acids in length for complexes with class I MHC proteins, and usually about 8 to 11 amino acids. The peptides will be from about 6 to 35 amino acids in length for complexes with class II MHC proteins, usually from about 10 to 20 amino acids. The peptides may have a sequence derived from a wide variety of proteins. In many cases it will be desirable to use peptides, which act as T cell epitopes. The epitope sequences from a number of antigens are known in the art. Alternatively, the epitope sequence may be empirically determined by isolating and sequencing peptides bound to native MHC proteins, by synthesis of a series of putative antigenic peptides from the target sequence, then assaying for T cell reactivity to the different peptides, or by producing a series of MHC-peptide complexes with these peptides and quantification the T cell binding. Preparation of peptides, including synthetic peptide synthesis, identifying sequences, and identifying relevant minimal antigenic sequences is known in the art. In any case, the peptide comprised in the oligomeric MHC complex is preferably substantially homogeneous, meaning that preferably at least 80% of the peptides are identical, more preferably at least 90% and most preferably at least 95%.

Preferably the peptide is selected from the group consisting of (a) a peptide against which there is a pre-existing T cell immune response in a patient, (b) a viral peptide e.g. occurring in the group of Epstein Barr Virus, Cytomegalovirus, Influenza Virus, (c) an immunogenic peptide of immunogens used in common vaccination procedures, and (d) a tumour specific peptide, a bacterial peptide, a parasitic peptide or any peptide which is exclusively or characteristically presented on the surface of diseased, infected or foreign cell.

Generally, the nomenclature used herein and the laboratory procedures in recombinant DNA technology described are those well known and commonly employed in the art. Standard techniques are used for DNA and RNA isolation, amplification, and cloning. Generally enzymatic reactions involving DNA ligase, DNA polymerase, restriction endonucleases and the like are performed according to the manufacturer's specifications, using enzyme buffers supplied by the manufacturer. These techniques and various other techniques are generally performed as known in the art. The nucleotide sequences for most suitable receptor-ligand pair members, such as for MHC molecules, complementary binding pair members and oligomer-forming coiled coil domains such as that of COMP are known in the art as discussed hereinbefore.

The DNA constructs will typically include an expression control DNA sequence, including naturally associated or heterologous promoter regions, operably linked to protein coding sequences. An expression cassette for expressing the peptides used in the complex of the invention may further include appropriate start and stop codons, leader sequences coding sequences and so on, depending on the chosen host. The expression cassette can be incorporated into a vector suitable to transform the chosen host and/or to maintain stable expression in said host.

The term “operably linked” as used herein refers to linkage of a promoter up-stream from one or more DNA sequences such that the promoter mediates transcription of the DNA sequences. Preferably, the expression control sequences will be those eukaryotic or non-eukaryotic promoter systems in vectors capable of transforming or transfecting desired eukaryotic or non-eukaryotic host cells. Once the vector has been incorporated into the appropriate host, the host is maintained under conditions suitable for high-level expression of the nucleotide sequences, and the collection and purification of the expressed proteins.

Receptor-ligand pair member-DNA sequences and DNA sequences for suitable oligomerising domains from an oligomer-forming coiled-coil protein, including that of COMP, can be isolated in accordance with well-known procedures from a variety of human or other cells. Traditionally, desired sequences are amplified from a suitable cDNA library, which is prepared from messenger RNA that is isolated from appropriate cell lines. Suitable source cells for the DNA sequences and host cells for expression and secretion can be obtained from a number of sources, such as the American Type Culture Collection (ATCC) or other commercial suppliers.

The nucleotide sequences or expression cassettes used to transfect the host cells can be modified according to standard techniques to yield chimeric molecules with a variety of desired properties. The molecules of the present invention can be readily designed and manufactured utilizing various recombinant DNA techniques well known to those skilled in the art. For example, the chains can vary from the naturally occurring sequence at the primary structure level by amino acid insertions, substitutions, deletions, and the like. These modifications can be used in a number of combinations to produce the final modified protein chain. In general, modifications of the genes encoding the chimeric molecule may be readily accomplished by a variety of well-known techniques, such as site-directed mutagenesis.

The amino acid sequence variants as discussed above can be prepared with various objectives in mind, including, where MHC molecules are concerned, increasing the affinity of the molecule for target T cells, increasing or decreasing the affinity of the molecule for the respective CD4 or CD8 co-receptors interacting with class I or class II MHC complexes, for facilitating purification and preparation of the components of the complex or the complex as a whole or for increasing the stability of the complex, in vivo and ex vivo. The variants will, however, typically exhibit the same or similar biological activity as naturally occurring versions of the naturally occurring receptor-ligand pair members in general and MHC molecules in particular.

In addition, preparation of the oligomer is a straightforward process. For therapeutic uses this may be carried out in mammalian cell culture, e.g. in CHO, COS, or human cell lines. Alternatively other expression systems can be used for expressing the molecules of the present invention, such as insect cell culture, including baculovirus and Drosophila melanogaster expression systems, yeast expression systems, or prokaryotic expression systems such as Escherichia coli (E. coli). If expression is performed in a prokaryotic expression system, either soluble expression, i.e. directed into the cytoplasm or periplasm, or insoluble expression, i.e. into inclusion bodies is possible.

A typical vector for E. coli would be one of the pET family of vectors which combine efficient expression from bacteriophage T7 RNA polymerase within an inducible lac operon-based system. The vectors containing the nucleotide segments of interest can be transferred into the host cell by well-known methods, depending on the type of cellular host. For example, transformation into chemical- or electro-competent cells is commonly utilised for prokaryotic cells, whereas calcium phosphate treatment, cationic liposomes, or electroporation may be used for other cellular hosts. Other methods used to transfect mammalian cells include the use of Polybrene, protoplast chimeric, micro-projectiles and microinjection.

The oligomeric receptor-ligand pair member proteins, their respective polypeptide subunits, where applicable, and the chimeric proteins, respectively, may be co-expressed and assembled as oligomeric complexes in the same cell. Alternatively these receptor-ligand pair monomer and the oligomeric core may be produced separately and allowed to associate in vitro. Here the receptor-ligand pair member monomers and the oligomeric cores would typically be formed in separate association or refolding reactions. These components may thereupon also be purified separately before binding to the oligomeric core to form the fully assembled complexes.

Where oligomeric MHC complexes are concerned, the advantage of association of separate components in vitro is that oligomeric MHC-peptide complexes can be obtained with very high peptide homogeneity, e.g. greater than 95% or even greater than 99%. Where the complexes are expressed as fully assembled molecules, the peptide of interest can be introduced into the complexes, either by culturing the expressing cells with medium containing the antigenic peptide of interest, or by exchanging the peptide of interest with peptides that have endogenously bound during expression in vitro, which is typically done by incubating the purified complex with excess peptide at low or high pH so as to open the antigen binding pocket (see e.g. WO 93/10220). The antigenic peptide can also be covalently bound using standard procedures such as photoaffinity labeling (see e.g. WO 93/10220).

Alternatively, the peptide may be directly linked or fused to the expression construct of the one of the MHC α or β chain subunits. A suitable linker between the peptide portion and the N terminus of the chimeric protein or its complementary α or β chain, such as a polyglycine repeat sequence may be provided. Including the peptide as a chimeric peptide in the expression construct will allow for expression and folding of the complete oligomeric complex, which is in this case a complete MHC-peptide oligomeric complex without further addition of antigenic peptide or peptide exchange.

Conditions that permit folding and association of the subunits and chimeric proteins in vitro are known in the art. Assembly of class I MHC peptide complexes as well as assembly of functional class II complexes is e.g. described in EP 812 331. As one example of permissive conditions, roughly equimolar amounts of solubilised α and β subunits are mixed in a solution of urea in the presence of an excess of antigenic peptide of interest. In the case of class II MHC molecules refolding is initiated by dilution or dialysis into a buffered solution without urea. Peptides are loaded into empty class II heterodimers at about pH 5 to 5.5 over about 1 to 3 days, followed by neutralization, concentration and buffer exchange. Oligomerisation of the complex should occur simultaneously with formation of the α-β-peptide complex.

The assembled complex of the invention, or, initially the separate components thereof, including the oligomeric core and the chimeric protein, can be purified according to standard procedures of the art, including ammonium sulphate precipitation, gel electrophoresis, column chromatography, including gel filtration chromatography, ion exchange chromatography, hydrophobic interaction chromatography, affinity chromatography, and the like.

The resulting oligomeric receptor-ligand pair member complex of the present invention may be used therapeutically or in developing and performing assay procedures, immuno-stainings, and the like. Where the invention concerns MHC molecules, therapeutic uses of the oligomeric MHC complexes of the present invention require identification of the MHC haplotypes and antigens useful in treating a particular disease. In addition it may be necessary to determine the tissue types of patients before therapeutic use of the complexes according to the present invention, which is, however, a standard procedure well known in the art. The present invention is particularly suitable for treatment of cancers, infectious diseases, autoimmune diseases, and prevention of transplant rejection. Based on knowledge of the pathogenesis of such disease and the results of studies in relevant animal models, one skilled in the art can identify and isolate the MHC haplotype and antigens associated with a variety of such diseases.

In a fourth aspect the present invention therefore relates to a pharmaceutical or diagnostic composition, comprising the above oligomeric receptor-ligand pair member complexes as defined above. Pharmaceutical compositions comprising the proteins are useful for, e.g. parenteral administration, i.e. subcutaneously, intramuscularly or intravenously. In addition, a number of new drug delivery approaches are being developed. The pharmaceutical compositions of the present invention are suitable for administration using these new methods, as well.

In this aspect of the invention the oligomeric receptor-ligand pair member complexes described here can be targeted to the surfaces of target cells in vivo and in vitro. Where, for example oligomeric MHC complexes are envisaged, these complexes can be used to stimulate either allospecific T cell responses against the target cells in question or generate peptide-antigen-specific T cell responses against the same MHC-peptide target on MHC-peptide targets presented on other cells.

The cell surface target molecules to which the oligomeric MHC complexes can bind may be tumour associated antigens, such as carcinoembryonic antigen, placental alkaline phosphatase, polymorphic epithelial mucin, human chorionic gonadotrophin, CD20, prostate specific antigen and others. Target specific monoclonal antibody expressing cell lines can be generated by methods well known in the art. Such cell lines can in turn be used as a cDNA source for cloning of the binding portions of these anti-bodies for fusion with the chimeric protein of the invention.

The target cell may be cultured in vitro where it can, e.g. be cultured with cytotoxic T cells in order to activate and proliferate antigen-specific or allospecific T cells. Alternatively the target cells may be occurring in the patient in vivo and the complexes are administered to the patient. Oligomeric MHC complexes of the invention can here also be targeted to cell types that do not possess co-stimulatory molecules the complexes may also be used in vitro or in vivo to ablate specific T cell responses, such as allospecific T cell responses in a transplantation setting or antigen-specific T cell responses in an autoimmune disease context by causing anergy of the specific T cell types.

Where the target cell is cultured in vitro, it may, for example, be co-cultured with T cells that have been already pre-selected according to their antigen specificity, e.g. by means of fluorescence activated cell sorting with fluorochrome labeled oligomeric MHC-peptide complexes. Hence the complexes of the present invention can be used for example to purify expand allospecific or antigen-specific T cells and re-introducing these T cells to a patient autologously after expansion and/or other manipulation in cell culture in various disease situations.

Where MHC-peptide complexes are envisaged by the invention, the target cell may be a tumour cell or any diseased of foreign cell, which should be eliminated in a patient, such as a leukemia cell, or other cancer cell, a cell infected with a virus, such as HIV, hepatitis B or C virus, human papilloma virus, or a microbe or parasite.

Preferably the MHC-peptide combination to be used will be capable of producing a strong immune response in the patient. Accordingly the MHC molecule to be used may be mismatched to the tissue type of the patient which will produce strong allospecific immune responses, or it will be a tissue matched MHC molecule containing a viral or microbial peptide to which the patient is likely to already have a good immune response. Such peptides may be preferably derived from endemic persistent infections Influenza virus, Epstein Barr Virus, Cytomegalovirus or peptide from an organism against which the patient has likely had a prior vaccination.

In another embodiment the target cell is used as an antigen-presenting vehicle. It could be a natural antigen-presenting cell (APC). Alternatively, it could be a substitute cell, which carries a surface marker in high copy number, such as a healthy CD20 positive B cell, which expresses a high number of copies of CD20 on its surface. In this case the oligomeric receptor-ligand pair member complex can be targeted to the high-copy number surface marker to cause highly multivalent presentation of receptor-ligand pair members on the cell surface, which generates a powerful antigen-presenting vehicle. In the case of an oligomeric MHC complex, the complex will preferably comprise an anti-genic peptide that is a viral peptide, bacterial peptide, parasitic, peptide or cancer specific peptide which is exclusively or typically presented by MHC molecules on the surface of diseased, malignant or foreign cells which are desirable to be eliminated in the patient. As a consequence, forming an antigen presenting vehicle with the oligomeric MHC complex of the invention as described above may be used in vivo or in vitro to generate an immune response against undesired cells which are different from the target cells.

The proliferation and differentiation of T cells stimulated with the complexes according to the invention may be improved and directed by simultaneously administering cytokines to the patient or adding such cytokines to the cell culture. Such cytokines may include interleukins, granulocyte monocyte colony stimulating factor (GM-CSF) and the like.

Compared to these prior art technologies of targeting oligomeric receptor-ligand pair member complexes to the cell surface of target cells the complexes of the present invention open the possibility of generating complexes with fully humanized content, very compact size, multimeric structure of both the target cell binding portion of the molecule and the receptor-ligand pair member presenting portion of the molecule which are each positioned advantageously on opposite ends of the oligomeric coiled-coil domain, which simultaneously affords high target binding affinity and high binding affinity to the complementary receptor-ligand pair member, e.g. where the receptor-ligand pair members in the complex are MHC molecules, to T cell receptors on the surface of T cells.

The compositions for parenteral administration will commonly comprise a solution of the oligomeric receptor-ligand pair member complexes dissolved in an acceptable carrier, preferably an aqueous carrier. A variety of aqueous carriers can be used, e.g. buffered water, 0.4% saline, 0.3% glycine and the like. These solutions are sterile and generally free of particulate matter. These compositions may be sterilised by conventional, well-known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of the chimeric protein in these formulations can vary widely, i.e. from less than about 1 pg/ml, usually at least about 0.1 mg/ml to as much as 10-100 mg/ml and will be selected primarily based on fluid volumes, viscosities, etc. in accordance with the particular mode of administration selected.

A typical pharmaceutical composition for intramuscular injection could be made up to contain 1 ml sterile buffered water, and 0.1 mg of oligomer complex protein. A typical composition for intravenous infusion could be made up to contain 250 ml of sterile Ringer's solution, and 10 mg of oligomer complex protein. Actual methods for preparing parenterally administrable compositions will be known or apparent to those skilled in the art.

The oligomeric receptor-ligand pair member complexes and the oligomeric core itself of this invention can be lyophilised for storage and reconstituted in a suitable carrier prior to use. This technique has been shown to be effective and commonly used lyophilization and reconstitution techniques can be employed. It will be appreciated by those skilled in the art that lyophilization and reconstitution can lead to varying degrees of activity loss and that use levels may have to be adjusted to compensate.

Where the complexes of the invention are MHC complexes and they are used for detecting or separating the T cells according to the specificity of their antigen receptor, as described hereinbefore, this may involve any known suitable technique. Suitable techniques are e.g. disclosed in EP 812 331, which is incorporated by reference herein. More in detail, the oligomeric MHC complexes of the invention can be used in labeling and detection of antigen specific T cells in suspension or other biological samples, such as in tissue samples, or may be used for separation of T cells e.g. when bound or immobilised on substrates, including paramagnetic or other beads as known to a skilled worker, or by flow cytometry.

Hence the complexes of the present invention can be used for example to purify and enrich T cells that are specific for a particular antigen and re-introducing these T cells to a patient autologously after expansion and/or other manipulation in cell culture in various disease situations. Alternatively T cells could be selectively depleted, e.g. in the case of an autoimmune disorder or other unwanted T cell immune responses.

The oligomeric receptor-ligand pair member complexes according to the present invention and can allow for improved drug potency, due to their enhanced affinity for their complementary receptor-ligand pair members, better serum half-life and also improved pharmacokinetics, due to increased size and molecular mass.

Due to the possibility of expressing the chimeric proteins and the peptide chains of the receptor-ligand pair member in the oligomeric receptor-ligand pair member complexes of the invention in non-eukaryotic systems they are further easy to make at high yields, and can be expressed as sub-components, which can subsequently be refolded with one another, optionally in the presence of a homogeneous population of antigenic peptide where MHC complexes are concerned.

These and other advantages are available to the skilled worker from the foregoing description. The following examples are given for the purpose of illustration only and shall not be construed as limiting the present invention in any way.

### EXAMPLES

In the following examples amino acid sequences are listed in single letter code.

### Example I

The following is a detailed example for constructing a pentameric HLA Class II MHC complex as shown in FIG. 2 wherein Class II MHC-peptide monomers provided with a PK tag are coupled to an oligomeric core via an anti-PKtag-scFv which is fused to the N-terminus of the oligomerising domain of COMP.

The pentameric Class II MHC-peptide complex comprises three independent polypeptide chain components each represented N-terminal to C-terminal:
1) Class II β chain: HLA-DRB1*0101T
2) Class II α chain-tag: HLA-DRA*0101T-linker1-(PK tag)
3) Chimeric protein: (anti-PK-scFv)-linker2-COMP-linker3-BP

Wherein
In 1 above: HLA-DRB1*0101T has the natural amino acid sequence for HLA-DRB1*0101 truncated after amino acid 198 of the mature peptide
In 2 above: HLA-DRA*0101T-(PK tag) has the natural amino acid sequence for HLA-DRA1*0101 truncated after amino acid 192 of the mature peptide followed by the linker amino acid sequence LE followed by the amino acid sequence of the PK tag: GKPIPNPLLGLDST [SEQ ID NO: 1].
In 3 above:
   a) anti-PK-scFv has the structure V_{H} (GGGGS)₄ V_{L} [SEQ ID NO: 5]. The sequences V_{H} and V_{L} are taken from those published in Hanke et al., Immunogenetics (1995) 42:442-443.
   b) Linker 2 and linker 3 both have the amino acid sequence

PQPQPKPQPKPEPET [SEQ ID NO: 6]
c) COMP consists of the amino acids 21-85 of COMP and
d) BP is has the amino acid SLNDIFEAQKIEWHE [SEQ ID NO: 7]

HLA-DR1-PK-peptide monomeric complexes DRB1*0101/DRA1*0101 are cloned, expressed and refolded in the presence of antigenic peptide and purified based on the methods described in Cameron et al., Journal of Immunological Methods (2002) 268:51-69.

The chimeric protein is cloned, expressed, assembled into a pentameric core and purified by column chromatography based on the methods of the specific examples for cloning, expressing, assembling and purifying the chimeric proteins of the non-prepublished international application PCT/EP03/09056 filed Aug. 14, 2003 and assigned to the present applicant which are suitably adapted by the practitioner of ordinary skill in the art.

After the assembly reaction and before purification by chromatography, the complexes are biotinylated by means of the biotinylating enzyme BirA, as described in EP 812 331.

HLA-DR1-PK-peptide monomeric complexes are then conjugated in 1:5 molar ratio to the pentameric core to yield HLA-DR1-peptide pentamers. Upon conjugation, the fully assembled pentameric complexes may optionally undergo a further round of purification according to standard techniques.

HLA-DR1-peptide pentamers can then be used, e.g. in flow cytometry to detect HLA-DR1-peptide specific T cells according to standard methods well known in the art. Binding of the HLA-DR1-peptide pentamers can be visualized, e.g. by secondary detection with streptavidin PE.

Alternatively the HLA-DR1-peptide pentamers can labeled with streptavidin-PE prior to the flow cytometry experiment, e.g. by conjugating the HLA-DR1-peptide pentamers to PE in a 1:1 to 1:5 molar ratio, whichever yields the most suitable combination of staining intensity and minimizing background staining.

### Example II

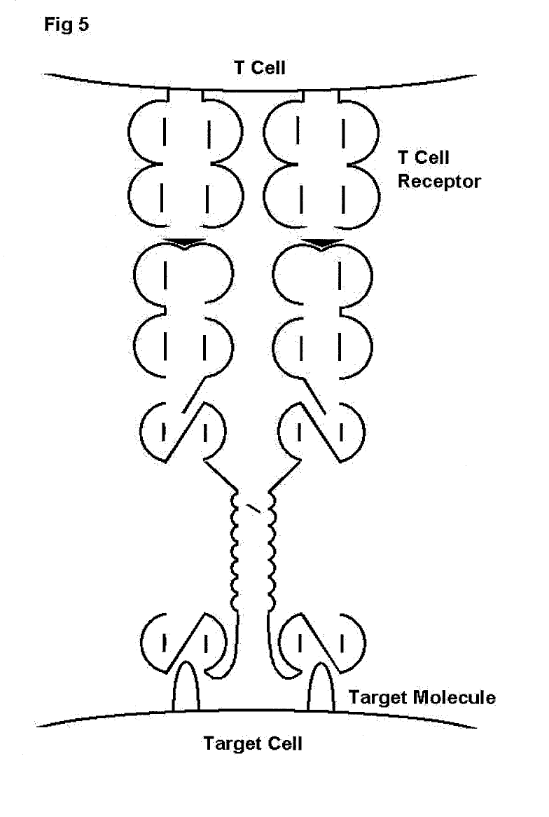

The following is a detailed example for constructing a pentameric HLA Class I MHC complex as shown in FIG. 5, which has an attachment means to bind to CD20 on the surface of B cells. Class I MHC-peptide monomers provided with a PK tag are coupled to an oligomeric core via an anti PKtag-scFv which is fused to the N-terminus of the oligomerising domain of COMP. Fused to the C-terminus of the COMP domain is an anti-CD20-scFv.

The pentameric Class I MHC-peptide complex comprises three independent polypeptide chain components each represented N-terminal to C-terminal:
1) Class I β chain: β2m-linker1-(PK tag)
   2) Class I α chain: HLA-A*0201T
3) Chimeric protein: (anti-PK-scFv)-linker2-COMP-linker3-(anti-CD20-scFv);

Wherein
In 1 above: β2m is the natural amino acid sequence of human β2 microglobulin followed by the amino acid linker sequence GGGGSG [SEQ ID NO: 8] followed by the amino acid sequence of the PK tag: GKPIPNPLLGLDST [SEQ ID NO: 1].
In 2 above: HLA-A*0201T has the natural amino acid sequence for HLA-A*0201 truncated after amino acid 276 of the mature peptide.
In 3 above:
   a) anti-PK-scFv has the structure V_{H} (GGGGS)₄ V_{L} [SEQ ID NO: 5]. The sequences V_{H} and V_{L} are taken from those published in Hanke et al., Immunogenetics (1995) 42:442-443;
   b) linker 2 and linker 3 both have the amino acid sequence PQPQPKPQPKPEPET [SEQ ID NO: 6];
   c) COMP consists of the amino acids 21-85 of COMP; and
   d) anti-CD20-scFv has the structure V_{H} (GGGGS)₅ V_{L} [SEQ ID NO: 4], as described in Schultz et al., Cancer Research (2000) 60:6663-6669.

HLA-A*0201-peptide monomeric complexes A*0201/β2m are cloned, expressed and refolded in the presence of antigenic peptide and purified based on the methods described in EP 812 331.

The chimeric protein is cloned, expressed, assembled into a pentameric core and purified by column chromatography based on the methods of the specific examples for cloning, expressing, assembling and purifying the chimeric proteins of the non-prepublished British national application GB 0323324.4 assigned to the present applicant which are suitably adapted by the practitioner of ordinary skill in the art.

HLA-A*0201-PK-peptide monomeric complexes are then conjugated in 1:5 molar ratio to the pentameric core to yield HLA-A*0201-peptide anti-CD20 pentamers. Upon conjugation, the fully assembled pentameric complexes may optionally undergo a further round of purification according to standard techniques and can then be used to for the targeting applications as described hereinbefore.

The present invention thus provides in its several aspects the following non-limiting, exemplary embodiments:
A. An oligomeric receptor-ligand pair member complex comprising
   (i) an oligomeric core, said core comprising at least two chimeric proteins, said chimeric proteins comprising a first section including at least one domain forming part of a first member of a complementary binding pair and a second section comprising an oligomerising domain derived from an oligomer-forming coiled-coil protein, wherein formation of the oligomeric core occurs by oligomerisation at the oligomerising domain of the chimeric proteins; and
   (ii) at least two receptor-ligand pair member peptides derived from a receptor-ligand pair member peptide chain or a functional part thereof, wherein each receptor-ligand pair member peptide further comprises attached thereto a second member of said complementary binding pair capable of binding to the first complementary binding pair member as defined in (i);
      and wherein each receptor-ligand pair member peptide is bound to the core via binding of the first and second members of the complementary binding pair;
      and in which complex at least two of the receptor-ligand pair member peptides are derived from the same receptor-ligand pair member peptide chain.
B. An oligomeric receptor-ligand pair member complex of A wherein the oligomerising domain comprised in the second section in at least one of the chimeric proteins is derived from the pentamerisation domain of the human cartilage oligomeric matrix protein (COMP).
C. An oligomeric receptor-ligand pair member complex of any one of the preceding listed embodiments wherein the first section in at least one of the chimeric proteins comprises one or more immunoglobulin-derived domains which are selected from the group consisting of an Fab fragment, a V_{L} domain, a V_{H} domain and a single chain variable fragment.
D. An oligomeric receptor-ligand pair member complex of any one of the preceding listed embodiments wherein the first section comprises a single chain variable fragment.
E. An oligomeric receptor-ligand pair member complex of D which additionally comprises the complementary variable (and constant) domains of respective immunoglobulin domains.
F. An oligomeric receptor-ligand pair member complex of any one of the preceding listed embodiments wherein at least one of the chimeric proteins further comprises a first linker between the first section and the second section.
G. An oligomeric receptor-ligand pair member complex of any one of the preceding listed embodiments wherein the complementary binding pair member as recognized by the binding pair member as defined in (i) is covalently attached to a receptor-ligand pair member peptide chain.
H. An oligomeric receptor-ligand pair member complex of G wherein the second member of the complementary binding pair is a peptide fused to the receptor-ligand pair member peptide, preferably at its C terminal end.
I. An oligomeric receptor-ligand pair member complex of any one of the preceding listed embodiments further comprising attaching means for selectively attaching said complex to a target cell.
J. An oligomeric receptor-ligand pair member complex of any one of the preceeding listed embodiments wherein said attaching means is comprised in a third section of at least one of the chimeric proteins of the complex.
K. An oligomeric receptor-ligand pair member complex of any one of the preceding listed embodiments wherein said attaching means comprises a linking polypeptide with high specific affinity for a target cell specific molecule on the surface of the target cell.
L. An oligomeric receptor-ligand pair member complex of K wherein said linking polypeptide is a single chain variable fragment derived from a monoclonal antibody.
M. An oligomeric receptor-ligand pair member complex of any one of the preceding listed embodiments wherein at least one of the chimeric proteins further comprises one or more domains selected from the group consisting of a further linker, a tagging domain and a purification domain.
N. An oligomeric receptor-ligand pair member of any one of the preceding listed embodiments wherein the first section of the chimeric protein is located N-terminal of the second section and the third section, if present, is located C-terminal of said second section.
O. An oligomeric receptor-ligand pair member complex of any one of the preceding listed embodiments wherein the chimeric protein has the structure
   scFv-linker-COMP-linker-AA
   wherein
   scFv is the single chain variable fragment, preferably V(H)-linker-V(L);
   COMP is the oligomerisation domain of COMP;
   linker means a peptide linker; and
   AA is a peptide selected from one or more domains selected from the group consisting of a further linker, a tagging domain, a purification domain and a linking polypeptide with high specific affinity for a target cell specific molecule on the surface of the target cell.
P. An oligomeric receptor-ligand member complex of any one of the preceding listed embodiments wherein least one of the peptides derived from the receptor-ligand pair member or functional part thereof is an MHC peptide or functional part thereof.
Q. An oligomeric receptor-ligand pair member complex of any one of the preceding listed embodiments wherein the at least on of the peptides derived from the receptor-ligand pair member or functional part thereof is derived from the extra-cellular part of the MHC class I or II a chain or the extra-cellular part of the MHC class I or II β chain.
R. An oligomeric receptor-ligand pair member complex according to P or Q further comprising complementary MHC peptide chains to form at least two functional MHC binding complexes.
S. An oligomeric receptor-ligand pair member complex according to R further comprising a peptide bound to the MHC portions of the complex in the groove formed by the MHC α1 and α2 domains for class I complexes or the MHC α1 and β1 domains for class II complexes.
T. An oligomeric receptor-ligand pair member complex according to S wherein the peptide is substantially homogeneous.
U. An oligomeric receptor-ligand pair member complex according to S or T wherein the peptide is selected from the group consisting of (a) a peptide against which there is a pre-existing T cell immune response in a patient, (b) a viral peptide e.g. occurring in the group of Epstein Barr Virus, Cytomegalovirus, Influenza Virus, (c) an immunogenic peptide of immunogens used in common vaccination procedures, and (d) a tumour specific peptide, a bacterial peptide, a parasitic peptide or any peptide which is exclusively or characteristically presented on the surface of diseased, infected or foreign cell.
V. An oligomeric receptor-ligand pair member complex according to any one of the preceding listed embodiments comprising a label.
W. Use of the complex of any one of A to V to amplify antigen-specific or allospecific T cells in vivo or in vitro.
X. A pharmaceutical or diagnostic composition, comprising an oligomeric receptor-ligand pair member complex according to any one of A to V, optionally in combination with a pharmaceutically acceptable carrier.
Y. Use of the oligomeric receptor-ligand binding pair member complex of any one of P to V for preparing a pharmaceutical composition for immunizing a patient against a disease or condition which is characterized by the presence in the patient's body of cells displaying disease associated MHC binding peptides on the surface thereof that are associated with the said disease or condition and wherein the oligomeric complex comprises binding peptides which are similar to or substantially the same as the said disease associated MHC binding peptides.
Z. Use of the oligomeric receptor-ligand pair member complex of any one of A to V for preparation of a pharmaceutical composition for causing an immune response in a patient to destroy unwanted target cells by directing an immune response against said target cells.
AA. A method of preparing an oligomeric receptor-ligand pair member complex according to any one of A to V, said method comprising the steps of:
   (a) constructing a vector comprising the recombinant expression cassette comprising a promoter sequence operably linked to a nucleotide sequence coding for a chimeric protein which chimeric protein comprises a first section comprising at least one domain forming part of a first member of a complementary binding pair and a second section comprising an oligomerising domain derived from an oligomer-forming coiled-coil protein, wherein oligomerisation occurs by alignment of at least two substantially identical versions of the polypeptide chain from which the oligomerising domain is derived; expressing said vector in an appropriate host, and recovering the expressed chimeric protein;
   (b) oligomerising said chimeric protein by alignment of the oligomerisation domains to form a complex core;
   (c) providing a receptor-ligand pair member peptide derived from an receptor-ligand pair member peptide chain or a functional part thereof, which receptor-ligand pair member peptide further comprises attached thereto a second member of the said complementary binding pair and capable of binding to the first complementary binding pair in the first section of the chimeric protein; and
   (d) attaching the receptor-ligand pair member peptide by binding of the first and second members of the complementary binding pair to the core.
BB. An oligomeric core for forming an oligomeric receptor-ligand pair member complex of any one of A to V wherein said core comprises at least two chimeric proteins, said chimeric proteins comprising a first section including at least one domain forming part of a first member of a complementary binding pair and a second section comprises an oligomerising domain derived from an oligomer-forming coiled-coil protein, wherein formation of the oligomeric core occurs by oligomerisation at the oligomerising domain of the chimeric proteins, wherein the second member of the complementary binding pair is selected from the group of a polypeptide epitope tag of less than 50 amino acids in length, a polypeptide incorporating a post-translational modification.
CC. A method of labeling and or detecting a cell population in a sample according to the specificity of a complementary receptor-ligand pair member present on the surface of cells in the cell population, the method comprising
   (i) combining an oligomeric receptor-ligand pair member complex according to any one of A to V and a suspension or biological sample comprising the cell population and
   (ii) detecting the presence of specific binding of said complex and the cells in said population.
DD. A method of separating a cell population in a sample according to the specificity of a complementary receptor-ligand pair member present on the surface of cells in the cell population, the method comprising
   (i) combining an oligomeric receptor-ligand pair member complex according to to any one of claims A to V and a suspension or biological sample comprising the cell population, and
   (ii) separating cells in the cell population bound to said complex from unbound cells.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation within the spirit and scope of the appended claims.

1.-34. (canceled)

35. An oligomeric receptor-ligand pair member complex comprising
(i) an oligomeric core, said core comprising at least two chimeric proteins, said chimeric proteins each comprising a first section including at least one domain forming part of a first member of a complementary binding pair and a second section comprising an oligomerising domain derived from an oligomer-forming coiled-coil protein, wherein formation of the oligomeric core occurs by oligomerisation at the oligomerising domain of the chimeric proteins; optionally further comprising a first linker between the first section and the second section on at least one of the chimeric proteins, and
(ii) at least two receptor-ligand pair member peptides derived from a receptor-ligand pair member peptide chain or a functional part thereof, wherein each receptor-ligand pair member peptide further comprises attached thereto a second member of said complementary binding pair capable of binding to the first complementary binding pair member as defined in (i);
and wherein each receptor-ligand pair member peptide is bound to the core via binding of the first and second members of the complementary binding pair;
and in which complex at least two of the receptor-ligand pair member peptides are derived from the same receptor-ligand pair member peptide chain.

36. The oligomeric receptor-ligand pair member complex of claim 35 wherein the oligomerising domain comprised in the second section in at least one of the chimeric proteins is derived from the pentamerisation domain of the human cartilage oligomeric matrix protein (COMP).

37. The oligomeric receptor-ligand pair member complex of claim 35 wherein the first section in at least one of the chimeric proteins comprises one or more immunoglobulin-derived domains which are selected from the group consisting of an Fab fragment, a V_{L} domain, a V_{H} domain and a single chain variable fragment.

38. The oligomeric receptor-ligand pair member complex of claim 35 wherein the first section comprises a single chain variable fragment, optionally further comprising complementary domains of respective immunoglobulin domains.

39. The oligomeric receptor-ligand pair member complex of claim 35 wherein the complementary binding pair member as recognized by the binding pair member as defined in (i) is covalently attached to a receptor-ligand pair member peptide chain.

40. The oligomeric receptor-ligand pair member complex of claim 39 wherein the second member of the complementary binding pair is a peptide fused to the receptor-ligand pair member peptide, preferably at its C terminal end.

41. The oligomeric receptor-ligand pair member complex of claim 35 further comprising attaching means for selectively attaching said complex to a target cell.

42. The oligomeric receptor-ligand pair member complex of claim 35 wherein at least one of the chimeric proteins further comprises one or more domains selected from the group consisting of a further linker, a tagging domain and a purification domain.

43. The oligomeric receptor-ligand pair member of claim 35 wherein the first section of the chimeric protein is located N-terminal of the second section and the third section, if present, is located C-terminal of said second section.

44. The oligomeric receptor-ligand pair member complex of claim 35 wherein the chimeric protein has the structure
scFv-linker-COMP-linker-AA
wherein
scFv is the single chain variable fragment, preferably V(H)-linker-V(L);
COMP is the oligomerisation domain of COMP;
linker means a peptide linker; and
AA is a peptide selected from one or more domains selected from the group consisting of a further linker, a tagging domain, a purification domain and a linking polypeptide with high specific affinity for a target cell specific molecule on the surface of the target cell.

45. The oligomeric receptor-ligand pair member complex of claim 35 wherein least one of the peptides derived from the receptor-ligand pair member or functional part thereof is an MHC peptide or functional part thereof.

46. The oligomeric receptor-ligand pair member complex according to claim 45 further comprising complementary MHC peptide chains to form at least two functional MHC binding complexes and optionally further comprising a peptide bound to the MHC portions of the complex in the groove formed by the MHC α1 and α2 domains for class I complexes or the MHC α1 and β1 domains for class II complexes.

47. The oligomeric receptor-ligand pair member complex according to claim 46 wherein the peptide is selected from the group consisting of (a) a peptide against which there is a pre-existing T cell immune response in a patient, (b) a viral peptide, and (c) a tumour specific peptide, a bacterial peptide, a parasitic peptide or any peptide which is exclusively or characteristically presented on the surface of diseased, infected or foreign cell.

48. A process comprising amplifying antigen-specific or allospecific T cells in vivo or in vitro using the complex of claim 46.

49. A pharmaceutical or diagnostic composition, comprising an oligomeric receptor-ligand pair member complex according to claim 35, optionally in combination with a pharmaceutically acceptable carrier.

50. A process comprising preparing a pharmaceutical composition for causing an immune response in a patient to destroy unwanted target cells by directing an immune response against said target cells, by employing the oligomeric receptor-ligand pair member complex of claim 46.

51. A method of preparing an oligomeric receptor-ligand pair member complex according to claim 35, said method comprising the steps of:
a) constructing a vector comprising the recombinant expression cassette comprising a promoter sequence operably linked to a nucleotide sequence coding for a chimeric protein which chimeric protein comprises a first section comprising at least one domain forming part of a first member of a complementary binding pair and a second section comprising an oligomerising domain derived from an oligomer-forming coiled-coil protein, wherein oligomerisation occurs by alignment of at least two substantially identical versions of the polypeptide chain from which the oligomerising domain is derived; expressing said vector in an appropriate host, and recovering the expressed chimeric protein;
b) oligomerising said chimeric protein by alignment of the oligomerisation domains to form a complex core;
c) providing a receptor-ligand pair member peptide derived from an receptor-ligand pair member peptide chain or a functional part thereof, which receptor-ligand pair member peptide further comprises attached thereto a second member of the said complementary binding pair and capable of binding to the first complementary binding pair in the first section of the chimeric protein; and
d) attaching the receptor-ligand pair member peptide by binding of the first and second members of the complementary binding pair to the core.

52. The oligomeric core for forming the oligomeric receptor-ligand pair member complex of claim 35 wherein said core comprises at least two chimeric proteins, said chimeric proteins comprising a first section including at least one domain forming part of a first member of a complementary binding pair and a second section comprises an oligomerising domain derived from an oligomer-forming coiled-coil protein, wherein formation of the oligomeric core occurs by oligomerisation at the oligomerising domain of the chimeric proteins, wherein the second member of the complementary binding pair is selected from the group of a polypeptide epitope tag of less than 50 amino acids in length, a polypeptide incorporating a post-translational modification.

53. A method of labeling and or detecting a cell population in a sample according to the specificity of a complementary receptor-ligand pair member present on the surface of cells in the cell population, the method comprising
(i) combining an oligomeric receptor-ligand pair member complex according to claim 35 and a suspension or biological sample comprising the cell population and
(ii) detecting the presence of specific binding of said complex and the cells in said population.

54. A method of separating a cell population in a sample according to the specificity of a complementary receptor-ligand pair member present on the surface of cells in the cell population, the method comprising
(i) combining an oligomeric receptor-ligand pair member complex according to claim 35 and a suspension or biological sample comprising the cell population, and (ii) separating cells in the cell population bound to said complex from unbound cells.


SEQUENCE LISTING (9 sequences)

SEQ ID NO: 1 - protein, 14 aa, Artificial
  Synthetic Construct
GKPIPNPLLGLDST

SEQ ID NO: 2 - protein, 10 aa, Artificial
  Synthetic Construct
EQKLISEEDL

SEQ ID NO: 3 - protein, 9 aa, Artificial
  Synthetic Construct
YPYDVPDYA

SEQ ID NO: 4 - protein, 25 aa, Artificial
  Synthetic Construct
GGGGSGGGGSGGGGSGGGGSGGGGS

SEQ ID NO: 5 - protein, 20 aa, Artificial
  Synthetic Construct
GGGGSGGGGSGGGGSGGGGS

SEQ ID NO: 6 - protein, 15 aa, Artificial
  Synthetic Construct
PQPQPKPQPKPEPET

SEQ ID NO: 7 - protein, 15 aa, Artificial
  Synthetic Construct
SLNDIFEAQKIEWHE

SEQ ID NO: 8 - protein, 6 aa, Artificial
  Synthetic Construct
GGGGSG

SEQ ID NO: 9 - protein, 64 aa, Unknown
  Sequence is from an unknown species of rat
QGQIPLGGDLAPQMLRELQETNAALQDVRELLRQQVKEITFLKNTVMECDACGMQPARTPGLSV